(19) United States
(12) Patent Application Publication
(10) Pub. No.: US 2026/0069374 A1
(43) Pub. Date: Mar. 12, 2026

(54) FASTENING DEVICE

(71) Applicant: Point Robotics Medtech Inc., Hsinchu County (TW)

(72) Inventor: KUN-PIN HUANG, Hsinchu County (TW)

(21) Appl. No.: 18/981,610

(22) Filed: Dec. 15, 2024

(30) Foreign Application Priority Data
Sep. 10, 2024 (TW) 113134147

(51) Int. Cl. A61B 90/00; A61B 90/50

(52) U.S. Cl. CPC A61B 90/39; A61B 90/50; A61B 2090/3983

(57) ABSTRACT

A fastening device including a fixing holder, a frame, and a locking member. The fixing holder includes a main base, a limiting block, and a supporting member. The main base includes a mounting portion that is disposed at a side thereof. The mounting portion has a recess. The limiting block is disposed in the recess. The limiting block has a limiting groove. The supporting member is connected to the main base and corresponds to the limiting block. The frame includes a limiting column, and the frame is assembled to the fixing holder through placing the limiting column within the limiting groove. The locking member is disposed at the supporting member and abuts the limiting column for fixing the frame in place.

### CROSS-REFERENCE TO RELATED PATENT APPLICATION

This application claims the benefit of priority to Taiwan Patent Application No. 113134147, filed on Sep. 10, 2024. The entire content of the above identified application is incorporated herein by reference.

### FIELD OF THE DISCLOSURE

The present disclosure relates to a fastening device, and more particularly to a fastening device applied to a marker for surgical navigation.

### BACKGROUND OF THE DISCLOSURE

When performing minimally invasive surgery, such as spinal surgery or laparoscopic surgery, a plurality of markers (e.g., reflective balls) need to be attached to surgical instruments and a surgical site on the patient's limb. These markers are used for generating coordinate signals for a surgical navigation system which guides the doctor to operate surgical instruments during surgery.

In the related art, the markers are fixed onto a frame which can be connected to a fixed base via connecting parts. Then, the fixed base is secured to positioning pins inserted previously into the patient's body. However, the adapter for fixing the markers is composed of multiple combining parts that need to be assembled before use. Therefore, the procedures of fixing marker frames and conducting surgical operation are a bit complicated, and it is difficult to stably secure the markers.

Therefore, how to overcome the above-mentioned problems through an improvement in structural design has become an important issue to be addressed in the related art.

### SUMMARY OF THE DISCLOSURE

In response to the above-referenced technical inadequacy, the present disclosure provides a fastening device which can be directly fixed onto a positioning pin without using other connecting parts and stably secure the markers, to solve the problem that the existing adapter for fixing marker frames needs to be assembled through complex assembling processes before use.

In order to solve the above-mentioned problems, one of the technical aspects adopted by the present disclosure is to provide a fastening device, which includes a fixing holder, a frame, and a locking member. The fixing holder includes a main base, a limiting block, and a supporting member. The main base includes a mounting portion that is disposed at a side thereof. The mounting portion has a recess. The limiting block is disposed in the recess. The limiting block has a limiting groove. The supporting member is connected to the main base and corresponds to the limiting block. The frame includes a limiting column, and the frame is assembled to the fixing holder through placing the limiting column in the limiting groove. The locking member is disposed at the supporting member and abuts the limiting column for fixing the frame.

According to the above, one advantage of the disclosure is that in the disclosed fastening device having an integrated design, the frame can be directly connected to the fixing holder, and the frame is then secured to a positioning pin via the fixing holder. In other words, the disclosed fastening device does not have to be assembled with other connecting components in advance, the overall structure of the fastening device has better stability and can be operated more easily.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of a fastening device according to an embodiment of the present disclosure;

FIG. 2 is a schematic exploded view of the fastening device according to the embodiment of the present disclosure;

FIG. 3 is a schematic view of a recess and a plurality of grooves of a main base according to the embodiment of the present disclosure;

FIG. 4 is a first schematic view of a frame and a limiting block of the fastening device according to the embodiment of the present disclosure;

FIG. 5 is a second schematic view of the frame and the limiting block of the fastening device according to the embodiment of the present disclosure;

FIG. 6 is a third schematic view of the frame and the limiting block of the fastening device according to the embodiment of the present disclosure;

FIG. 7 is a schematic cross-sectional view taken along line VII-VII of FIG. 1;

FIG. 8 is a schematic cross-sectional view of a washer according to the embodiment of the present disclosure; and

FIG. 9 is another schematic cross-sectional view taken along line VII-VII of FIG. 1.

### DETAILED DESCRIPTION OF THE EXEMPLARY EMBODIMENTS

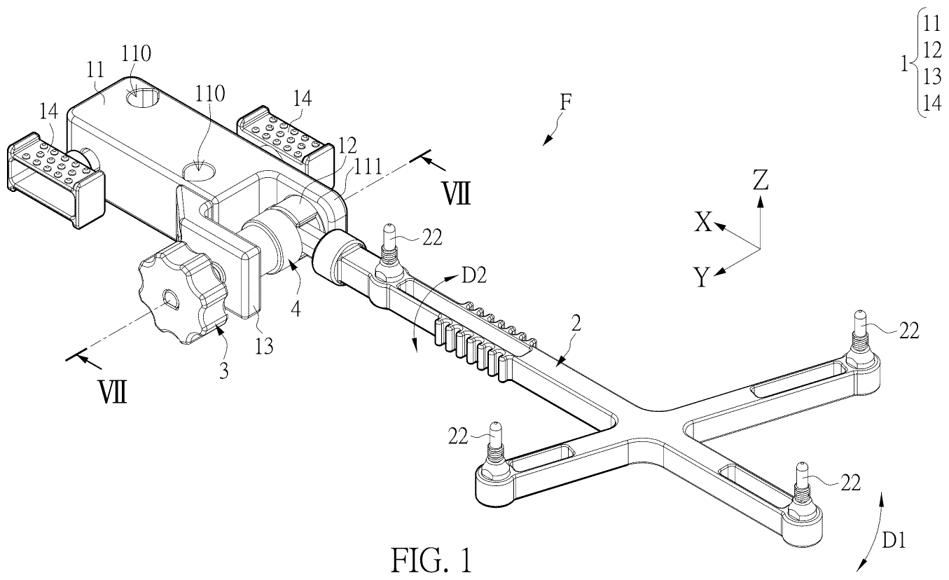

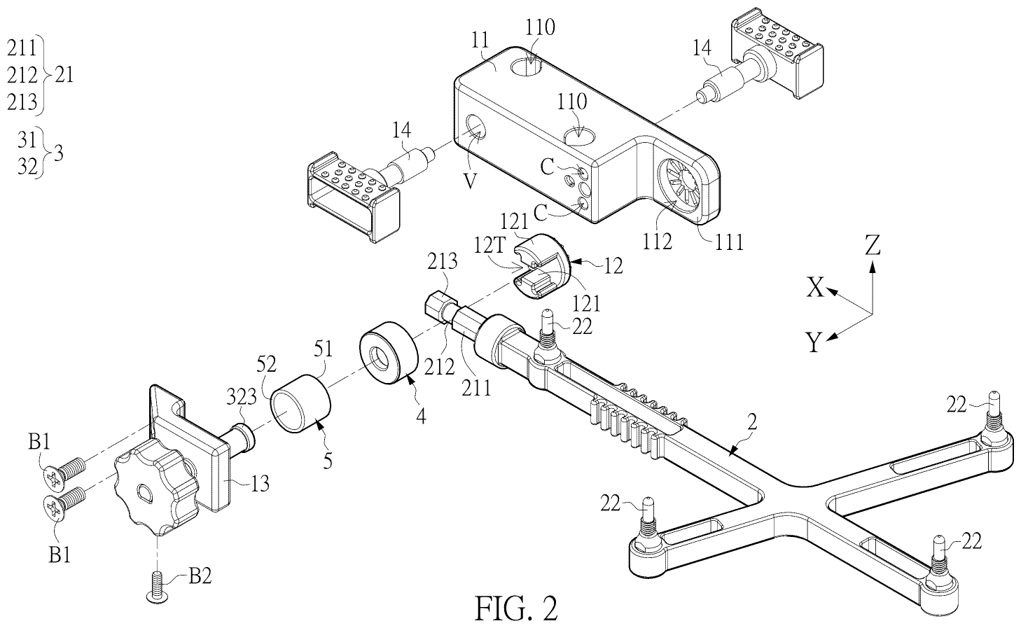

Reference is made to FIG. 1 and FIG. 2. An embodiment of the present disclosure provides a fastening device F, which includes a fixing holder 1, a frame 2, and a locking member 3. The fixing holder 1 includes a main base 11, a limiting block 12, and a supporting member 13. The main base 11 includes a mounting portion 111. The mounting portion 111 is disposed at a side of the main base 11. The mounting portion 111 has a recess 112. The limiting block 12 is disposed in the recess 112. The limiting block 12 includes two sidewalls 121, and the limiting block 12 has a limiting groove 12T that is formed between the two sidewalls 121. The supporting member 13 is connected to the main base 11. The supporting member 13 and the mounting portion 111 are located at the same side of the main base 11. The supporting member 13 corresponds to the limiting block 12, and the limiting block 12 is located between the mounting portion 111 and the supporting member 13. The locking member 3 is movably disposed at the supporting member 13. As shown in FIG. 2, the locking member 3 can reciprocate along an axis (e.g., a Y-axis).

The main base 11 has a plurality of threaded holes C. The supporting member 13 can be fixed to the main base 11 by screwing a plurality of screws B1 into the plurality of threaded holes C. The method of fixing the supporting member 13 to the main base 11 is not limited in the present disclosure. The frame 2 includes a limiting column 21. The limiting column 21 of the frame 2 can be disposed in the limiting groove 12T, so the frame 2 can be assembled with the fixing holder 1. The locking member 3 abuts the limiting column 21 to fix the frame 2 in place. In addition, the frame 2 includes a plurality of prongs, each of the prongs is provided with a fixing pin 22. In the present disclosure, the frame 2 is a dynamic reference frame (DRF), which can carry a plurality of markers such as reflective balls (not shown in the figures) through a plurality of fixing pins 22.

Reference is further made to FIG. 1 and FIG. 2. The main base 11 further includes a plurality of mounting holes 110 and a plurality of through holes V. The mounting holes 110 and the through holes V can be located on different sides of the main base 11. The mounting holes 110 respectively communicate with the through holes V. The fixing holder 1 further includes a plurality of fixing members 14. When the fixing members 14 are being used, a positioning pin (not shown in the figures) that has been inserted into a body part, such as a bone, passes through one of the mounting holes 110, and one of the fixing members 14 is inserted into a corresponding one of the through holes V to abut against the positioning pin. By tightly screwing the fixing members 14 to exert force on the positioning pin, the fixing holder 1 and the frame 2 are thus fixed to the positioning pin. Furthermore, the markers on the frame 2 can provide a coordinate reference for a surgical navigation system, medical personnel can operate surgical instruments under guiding of the surgical navigation system.

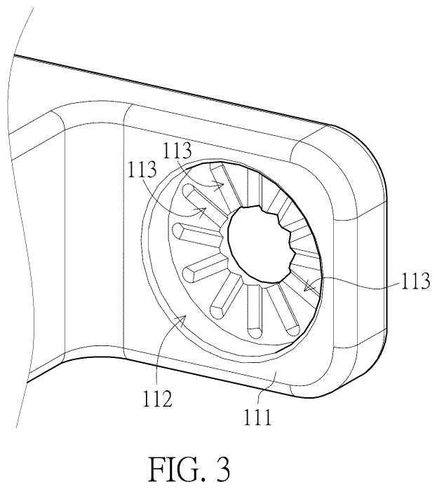

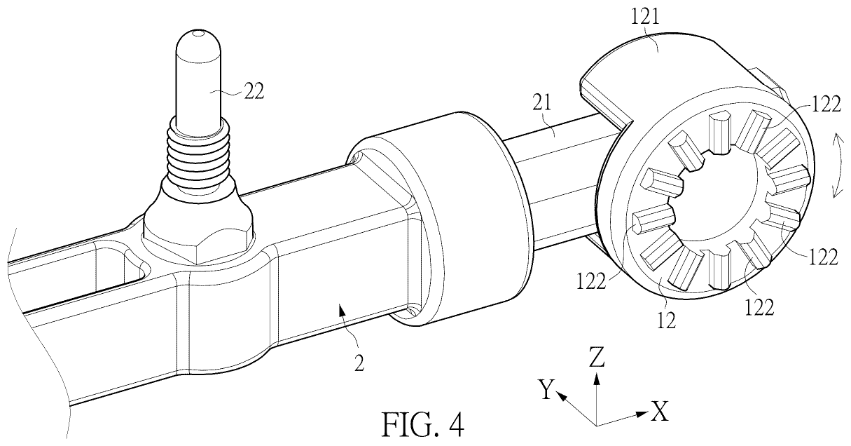

Reference is made to FIGS. 2, 3, and 4. At a side adjacent to the recess 112, the limiting block 12 includes a plurality of protruding portions 122. A bottom surface of the recess 112 has a plurality of grooves 113, and the plurality of grooves 113 corresponds to the plurality of protruding portions 122, respectively. Specifically, the plurality of protruding portions 122 and the plurality of grooves 113 are arranged at fixed intervals and in a radial pattern.

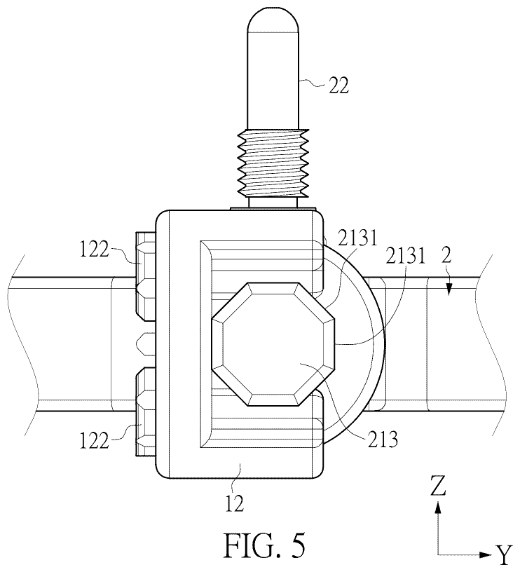

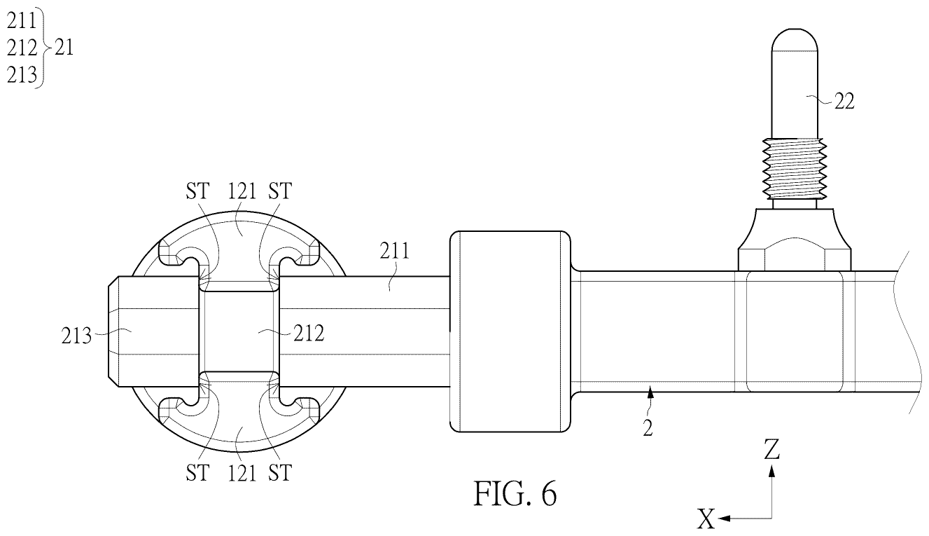

Reference is made to FIG. 5 and FIG. 6. The limiting column 21 includes a first part 211, a second part 212, and a third part 213. The second part 212 is disposed between the first part 211 and the third part 213. A shape of the second part 212 is different from shapes of the first part 211 and the third part 213. For example, the shapes of the first part 211 and the third part 213 are polygonal prisms, and the shape of the second part 212 is a cylinder. A diameter of the cylinder (i.e., the second part 212) is less than a width of each of the polygonal prisms (i.e., the first part 211 and the third part 213). Therefore, a step riser ST is formed between the second part 212 and the first part 211, and another step riser ST is formed between the second part 212 and the third part 213.

As shown in FIG. 6, when the limiting column 21 of the frame 2 is assembled with the limiting block 12, the second part 212 of the limiting column 21 is engaged into the limiting groove 12T. The two sidewalls 121 of the limiting block 12 are used to limit a position of the frame 2 along a first direction (a Z-axis direction). Moreover, the first part 211 and the third part 213 abut against the two sidewalls 121 through the step risers ST, so as to limit a position of frame 2 along a second direction (an X-axis direction). The first direction is perpendicular to the second direction.

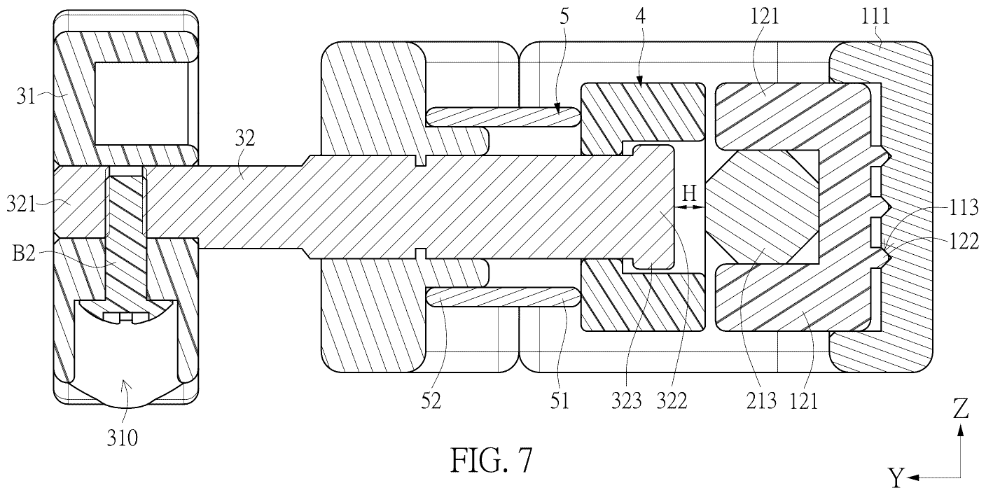

Reference is made to FIG. 2 and FIG. 7. The fastening device F further includes a washer 4 and an elastic member 5. The washer 4 is disposed between the limiting block 12 and the supporting member 13. The elastic member 5 is disposed between the washer 4 and the supporting member 13. As shown in FIG. 7, a first end 51 of the elastic member 5 abuts against the washer 4, and a second end 52 of the elastic member 5 abuts against the supporting member 13. Thereby, compressing the elastic member 5 will induce an elastic force to compel the washer 4 to abut against the two sidewalls 121 of the limiting block 12, and then push the limiting block 12 to fit in the recess 112. Furthermore, the plurality of protruding portions 122 of the limiting block 12 then engage in the plurality of grooves 113 of the mounting portion 111, respectively.

Reference is further made to 1. The frame 2 can rotate along a first rotation direction D1 with the limiting block 12 as the pivot and the Y-axis direction as the axial direction. When the frame 2 rotates, the plurality of protruding portions 122 and the plurality of grooves 113 move relative to each other and are misaligned. Through the design of the protruding portions 122 and the grooves 113, a user can easily control the rotation angle of frame 2. Regardless of the direction to which the frame 2 is rotated, the frame 2 is firmly secured, and it is easy to occur angle deviation.

In addition, in the present disclosure, the elastic force exerted by the elastic member 5 can be maintained at a constant value without manual adjustment. The constant elastic force exerted by the elastic member 5 can appropriately limit the plurality of protruding portions 122 in the plurality of grooves 113, so the user can smoothly rotate the frame 2 without exerting too much force. In other words, the elastic force exerted by the elastic member 5 does not cause excessive interference between the protruding portions 122 and the grooves 113, and the user can easily rotate the frame 2.

Compared to a conventional adapter, an elastic force is generated by turning a knob and a shaft to compress a spring. Furthermore, the elastic force of the spring is not a fixed value, a user needs to manually release the knob and shaft for controlling the elastic force of the spring to the extent that the frame with reflective balls can be rotated. As a result, the elastic force of the spring may not be the same every time the user manually turns the knob, and the user cannot adjust the elastic force consistently.

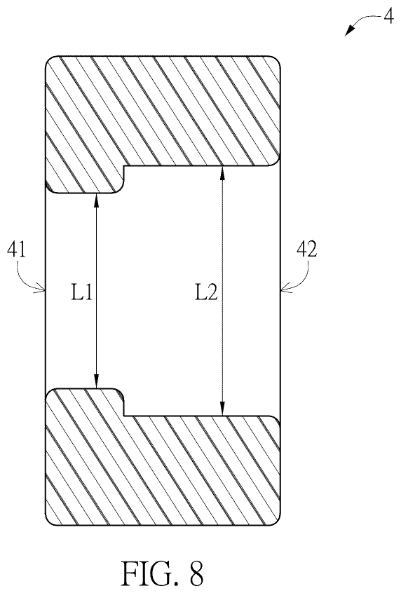

Reference is made to FIG. 7 to FIG. 9. The locking member 3 includes a knob 31 and a shaft 32. The washer 4 and the elastic member 5 are sleeved on the shaft 32. As shown in FIG. 2, the shaft 32 is perpendicular to the limiting column 21 of the frame 2. The shaft 32 has a first end 321 and a second end 322. The knob 31 is disposed at the first end 321, and the second end 322 includes a flange portion 323. As shown in FIG. 8, the washer 4 includes a first opening 41 and a second opening 42 which communicate with the first opening 41. An aperture L1 of the first opening 41 is smaller than an aperture L2 of the second opening 42. The width of the cross-section at the second end 322 with the flange portion 323 is greater than the aperture L1 but smaller than the aperture L2. When the washer 4 is sleeved on the shaft 32, the first opening 41 faces toward the elastic member 5, and the second opening 42 faces toward the limiting block 12.

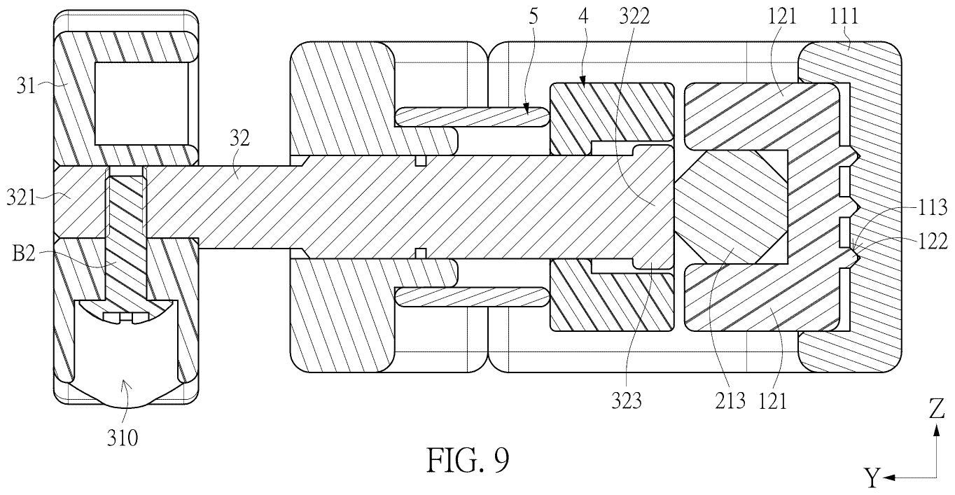

FIG. 7 and FIG. 9 show states of the shaft 32 in different positions. In FIG. 7, the shaft 32 does not contact the limiting column 21 of the frame 2. A distance H exists between the second end 322 of the shaft 32 and the first part 211 or the third part 213 of the limiting column 21. In FIG. 9, the knob 31 is turned (e.g., in a clockwise direction) to drive the shaft 32 to move toward the limiting column 21, such that the second end 322 of the shaft 32 moves by a distance that is approximately equal to the distance H, and the second end 322 thus abuts against the limiting column 21. In other words, the distance H is a stroke of the shaft 32. After the shaft 32 abuts against the limiting column 21, the shaft 32 is fixed by inserting a screw B2 into a fixing hole 310 of the knob 31.

Specifically, since the diameter of the cylinder (i.e., the second part 212) of the limiting column 21 is smaller than the width of the polygonal prism (i.e., the first part 211 and the third part 213), the second end 322 of the shaft 32 abuts against the sides of the first part 211 and the third part 213 (e.g., a side 2131 of the third part 213 shown in FIG. 5).

Reference is further made to FIGS. 1 and 5. Preferably, the first part 211 and the third part 213 are hexagonal prisms. As shown in FIG. 5, through structural design of the hexagonal prisms, shapes of the first part 211 and the third part 213 of the limiting column 21 resemble the shape of a cylinder, and the user may effortlessly rotate the frame 2 around the X-axis (in a second rotation direction D2) in sections. Therefore, the frame 2 has two degrees of freedom, and can rotate in the first rotation direction D1 and spin in the second rotation direction D2.

Moreover, the structural design of the hexagonal prism makes contact between the limiting column 21 and the limiting block 12 (as well as contact between the limiting column 21 and the shaft 32) as line contact when the frame spins. So, it is more convenient for the user to control a spin angle of the frame 2. The frame 2 can be firmly secured regardless of the direction to which the frame 2 is rotated, and it is easy to occur angle deviation.

On the other hand, when the rotation angle or spin angle of the frame 2 needs to be readjusted, the screw B2 can be loosened and taken out, and then the knob 31 is turned in another rotation direction (e.g., a counterclockwise direction) to drive the shaft 32 to move away from the limiting column 21. Furthermore, when the shaft 32 is driven to move by the distance H, the flange portion 323 around the second end 322 abuts against an interior of the washer 4 that is located at a peripheral edge of the first opening 41, so as to prevent the shaft 32 from detaching from the washer 4 when the user exerts an excessive force.

### Beneficial Effects of the Embodiment

In the disclosed fastening device F, which has an integrated design, the frame 2 can be directly connected to the fixing holder 1, and the frame 2 is then secured to the positioning pin via the fixing holder 1. As a result, the fastening device F does not have to be assembled with other connecting components in advance, the overall structure of the fastening device has better stability and can be operated more easily.

Moreover, the disclosed fastening device F utilizes the elastic member 5 to generate the elastic force to limit the limiting block 12 in the recess 112. The elastic force exerted by the elastic member 5 can be maintained at a constant value without manual adjustment. The constant elastic force exerted by the elastic member 5 can appropriately limit the plurality of protruding portions 122 in the plurality of grooves 113, so the user can smoothly rotate the frame 2 without exerting too much force. In other words, the elastic force exerted by the elastic member 5 does not cause excessive interference between the protruding portions 122 and the grooves 113, and the user can easily rotate the frame 2.

Moreover, the first part 211 and the third part 213 are hexagonal prisms. Through structural design of the hexagonal prism, shapes of the first part 211 and the third part 213 of the limiting column 21 approximate to the shape of a cylinder, the user may effortlessly spin the frame 2 around the X-axis (in a second rotation direction D2) in sections.

In addition, contact between the limiting column 21 and the limiting block 12, as well as between the limiting column 21 and the shaft 32, are as line contact when the frame spins. So, it is more convenient for the user to control a spin angle of the frame 2. The frame 2 can be firmly secured regardless of the direction to which the frame 2 is rotated, and it is easy to occur angle deviation.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A fastening device, comprising:
a fixing holder including:
a main base including a mounting portion that is disposed at a side thereof, the mounting portion having a recess;
a limiting block disposed in the recess, wherein the limiting block has a limiting groove; and
a supporting member connected to the main base and corresponding to the limiting block;
a frame including a limiting column, wherein the frame is assembled to the fixing holder through placing the limiting column within the limiting groove; and
a locking member disposed at the supporting member, wherein the locking member abuts the limiting column for fixing the frame.

2. The fastening device according to claim 1, wherein the limiting block includes two sidewalls, and the limiting groove is located between the two sidewalls.

3. The fastening device according to claim 2, further comprising a washer that is disposed between the limiting block and the supporting member.

4. The fastening device according to claim 3, further comprising an elastic member that is disposed between the washer and the supporting member, wherein one end of the elastic member abuts against the washer, and the other end of the elastic member abuts against the supporting member.

5. The fastening device according to claim 4, wherein the limiting block includes a plurality of protruding portions which are adjacent to a side of the recess, and a bottom surface of the recess has a plurality of grooves that corresponds to the plurality of protruding portions.

6. The fastening device according to claim 5, wherein the elastic member is used to provide an elastic force to the washer and push the washer to abut against the two sidewalls of the limiting block, such that the plurality of protruding portions are engaged in the plurality of grooves, respectively.

7. The fastening device according to claim 4, wherein the limiting column includes a first part, a second part, and a third part, the second part is disposed between the first part and the third part, a shape of the second part is different from shapes of the first part and the third part, the shapes of the first part and the third part are polygonal prisms, the shape of the second part is a cylinder, and a diameter of the cylinder is less than a width of each of the polygonal prisms.

8. The fastening device according to claim 7, wherein two step risers are respectively formed between the second part and the first part and between the second part and the third part, the second part of the limiting column is engaged in the limiting groove, and the two step risers respectively abut the two sidewalls of the limiting block, such that positions of the frame along a first direction and a second direction are restricted, wherein the first direction is perpendicular to the second direction.

9. The fastening device according to claim 7, wherein the locking member includes a knob and a shaft, the elastic member and the washer are sleeved on the shaft, the shaft includes a first end and a second end, the knob is disposed at the first end, and the second end includes a flange portion; wherein the washer includes a first opening and a second opening that are communicated with each other, the first opening faces toward the elastic member, the second opening faces toward the limiting block, and an aperture of the first opening is smaller than an aperture of the second opening; wherein the knob can be rotated to drive the shaft to move toward the limiting column, such that the second end abuts against the first part of the limiting column.

10. The fastening device according to claim 9, wherein the knob is configured to be rotated to drive the shaft to move away from the limiting column, such that the flange portion of the second end abuts against an interior of the washer at a peripheral edge of the first opening.
